(19) United States
(12) Patent Application Publication
(10) Pub. No.: US 2010/0318159 A1
(43) Pub. Date: Dec. 16, 2010

(54) MINIATURE REMOTE CONTROLLER FOR IMPLANTABLE MEDICAL DEVICE

(75) Inventors: Daniel Aghassian, Glendale, CA (US); Md. Mizanur Rahman, Stevenson Ranch, CA (US)

Correspondence Address: Wong, Cabello, Lutsch, Rutherford & Brucculeri LLP, 20333 SH 249, Suite 600, Houston, TX, 77070, US

(73) Assignee: Boston Scientific Neuromodulation Corporation, Valencia, CA (US)

(21) Appl. No.: 12/484,052

(22) Filed: Jun. 12, 2009

(51) Int. Cl. A61N 1/08

(52) U.S. Cl. 607/59; 607/60

(57) ABSTRACT

A miniature remote controller for an implantable medical device provides a subset of the functionality of a full-sized remote controller for the implantable medical device. The two remote controllers each have a user interface, which can be different from each other. A remote controller for an implantable medical device can have a coil for communicating with the implantable medical device, where the coil is wrapped around a coil axis parallel to a long axis of a housing of the remote controller. A user interface of the remote controller can have an indicator light to indicate success or failure of a communication with the implantable medical device and status of the implantable medical device. The housing of the remote controller can have two differently sized sections.

### TECHNICAL FIELD

The present invention relates to the field of implantable medical devices, and in particular to a remote control for implantable medical devices.

### BACKGROUND ART

Implantable stimulation devices are devices that generate and deliver electrical stimuli to body nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder sublaxation, etc. The present invention may find applicability in all such applications, although the description that follows will generally focus on the use of the invention within a Spinal Cord Stimulation (SCS) system, such as that disclosed in U.S. Pat. No. 6,516,227, which is incorporated herein by reference in its entirety.

Spinal cord stimulation is a well-accepted clinical method for reducing pain in certain populations of patients. As shown in FIG. 1, a SCS system typically includes an Implantable Pulse Generator (IPG) 100, which includes a biocompatible case 30 formed of titanium for example. The case 30 typically holds the circuitry and power source or battery necessary for the IPG 100 to function, although IPGs can also be powered via external RF energy and without a battery. The IPG 100 is coupled to electrodes 106 via one or more electrode leads (two such leads 102 and 104 are shown), such that the electrodes 106 form an electrode array 110. The electrodes 106 are carried on a flexible body 108, which also houses the individual signal wires 112 and 114 coupled to each electrode. In the illustrated embodiment, there are eight electrodes on lead 102, labeled E1-E8, and eight electrodes on lead 104, labeled E9-E16, although the number of leads and electrodes is application specific and therefore can vary.

FIG. 2 shows portions of an IPG system in cross section, including the IPG 100 and a remote controller 12. The IPG 100 typically includes an electronic substrate assembly 14 including a printed circuit board (PCB) 16, along with various electronic components 20, such as microprocessors, integrated circuits, and capacitors mounted to the PCB 16. Two coils are generally present in the IPG 100: a telemetry coil 13 used to transmit/receive data to/from the remote controller 12, and a charging coil 18 for charging or recharging the IPG's power source or battery 26 using an external charger (not shown). The telemetry coil 13 can be mounted within the header connector 36 as shown.

As just noted, a remote controller 12, such as a hand-held, is used to wirelessly send data to and receive data from the IPG 100. For example, the remote controller 12 can send programming data to the IPG 100 to set the therapy the IPG 100 will provide to the patient. In addition, the remote controller 12 can act as a receiver of data from the IPG 100, receiving various data reporting on the IPG's status.

The communication of data to and from the remote controller 12 occurs via magnetic inductive coupling. When data is to be sent from the remote controller 12 to the IPG 100, coil 17 is energized with an alternating current (AC). Such energizing of the coil 17 to transfer data can occur using a Frequency Shift Keying (FSK) protocol for example, such as disclosed in U.S. patent application Ser. No. 11/780,369, filed Jul. 19, 2007. Energizing the coil 17 generates an electromagnetic field, which in turn induces a current in the IPG's telemetry coil 13, which current can then be demodulated to recover the original data.

As is well known, inductive transmission of data or power occurs transcutaneously, i.e., through the patient's tissue 25, making it particular useful in a medical implantable device system.

Remote controllers available today are bulky and inconvenient for many patients. An example remote controller 12 is shown in FIG. 3A. The remote controller 12 often contains a display 265, such as an LCD display, for indicating information to the patient. The remote controller 12 often also has numerous buttons to allow control over the IPG 100, such as buttons 270, 272, 274, and 276, as well as ports (not shown) for connecting the remote controller 12 to a power source or a programming source. All of these features tend to increase the size and weight of the remote controller 12.

FIGS. 3B and C are alternate views of the conventional remote controller 12 of FIG. 3A, with some or all of the housing removed to show internal components of the remote controller 12. FIG. 3B is a bottom view of the remote controller 12, showing a non-replaceable battery 126, a printed circuit board (PCB) 120, and the coil 17. As can be seen in FIG. 3B, the coil 17 is an air core coil, and is placed below the PCB 120. The coil 17 is wound around an axis 99 perpendicular to a long axis 101 of the remote controller 12. In addition, as can be seen in FIGS. 3B/C, the axis 99 extends perpendicularly through the PCB 120, which covers the entire coil 17.

### SUMMARY OF INVENTION

A miniature remote controller for an implantable medical device provides a subset of the functionality of a full-sized remote controller for the implantable medical device. The miniaturized remote controller and the full-sized remote controller each have a user interface, which can be different from each other.

In one embodiment, a remote controller for an implantable medical device has a coil for communicating with the implantable medical device, where the coil is wrapped around a coil axis parallel to a long axis of a housing of the remote controller. In another embodiment, a user interface of a remote controller for an implantable medical device comprises an indicator light to indicate success or failure of a communication with the implantable medical device.

In another embodiment, a housing of a remote controller for an implantable medical device has two differently sized sections, and a coil within the housing is wrapped around a coil axis parallel to a long axis of the housing.

### BRIEF DESCRIPTION OF DRAWINGS

FIG. 1 illustrates conventional implantable medical devices according to the prior art;

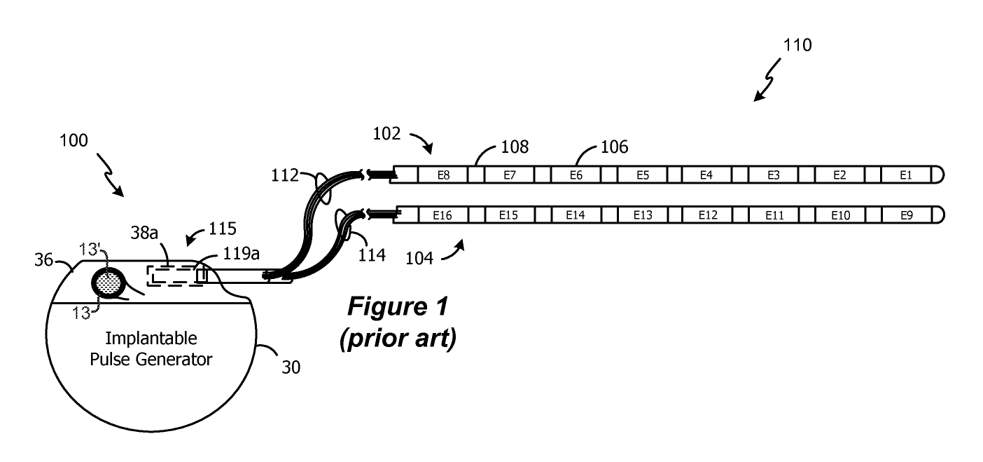

FIG. 2 illustrates the use of a remote controller to communicate with an implantable medical device according to the prior art;

FIGS. 3A, B, and C illustrate a full sized remote controller for an implantable medical device according to the prior art;

FIGS. 4A and B are block diagrams illustrating an embodiment of a miniaturized limited function remote controller according to one embodiment;

FIGS. 5A-C are block diagrams illustrating certain features of the miniaturized limited function remote controller of FIG. 4;

FIG. 6A is a block diagram illustrating elements of a miniaturized remote controller according to one embodiment;

FIG. 6B is a block diagram illustrating elements of a miniaturized remote controller according to another embodiment;

FIG. 7 is a block diagram illustrating another embodiment of a miniaturized remote controller;

FIGS. 8A/B are block diagrams illustrating two views of yet another embodiment of a miniaturized remote controller; and

FIG. 9A is a block diagram illustrating the relative orientation of one embodiment of a miniaturized remote controller and an IPG; and

FIG. 9B is a block diagram illustrating the relative orientation of a conventional remote controller and an IPG.

### DESCRIPTION OF EMBODIMENTS

The description that follows relates to use of the invention within a spinal cord stimulation (SCS) system. However, the invention is not so limited. Rather, the invention may be used with any type of implantable medical device system that could benefit from improved coupling between an external device and the implanted device. For example, the present invention may be used as part of a system employing an implantable sensor, an implantable pump, a pacemaker, a defibrillator, a cochlear stimulator, a retinal stimulator, a stimulator configured to produce coordinated limb movement, a cortical and deep brain stimulator, or in any other neural stimulator configured to treat any of a variety of conditions.

Patients with implanted neurostimulators use the remote controller (RC) 12 for communicating and controlling their implant. Typically different stimulation settings are needed to provide complete pain coverage throughout the day. The RC 12 is used by the patient to adjust the stimulator output to obtain the best therapy. Different therapy settings may be required for when the patient is sleeping, standing, sitting, or driving. Some settings are saved as “presets” or “programs” and can be selected by the patient using the RC 12. Common use of the RC 12 is to increase or decrease the strength, select different areas of the body to be stimulated, select between presets, and to shut off and turn on stimulation.

Remote controllers available today are bulky and inconvenient for many patients. A miniature remote controller that can be carried conveniently and discretely would be a significant benefit and convenience to patients.

At the same time, there are practical constraints that can limit the miniaturization that patients would find usable. Many patents are elderly or otherwise not in good health, and often find operation of small devices difficult or impossible. Indeed, some patients find merely holding a small object difficult, much less manipulating small buttons or switches.

In addition, because the remote controller is battery powered, the housing of the remote controller must be big enough to hold a battery of sufficient power to operate the remote controller transmitter and receiver, in addition to the electronics necessary for the transmitter and receiver.

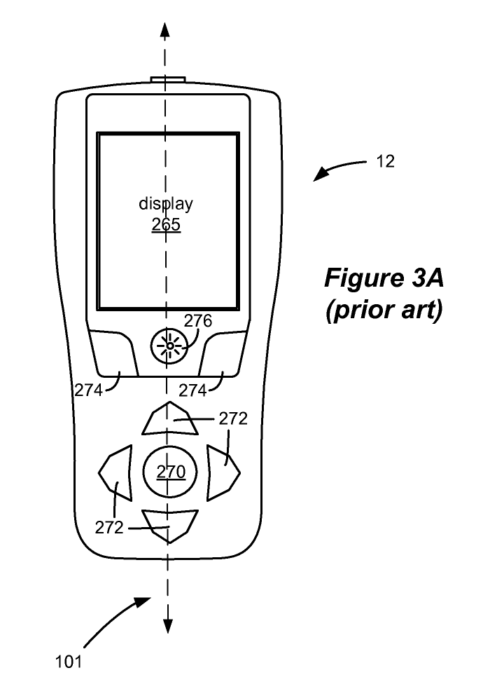

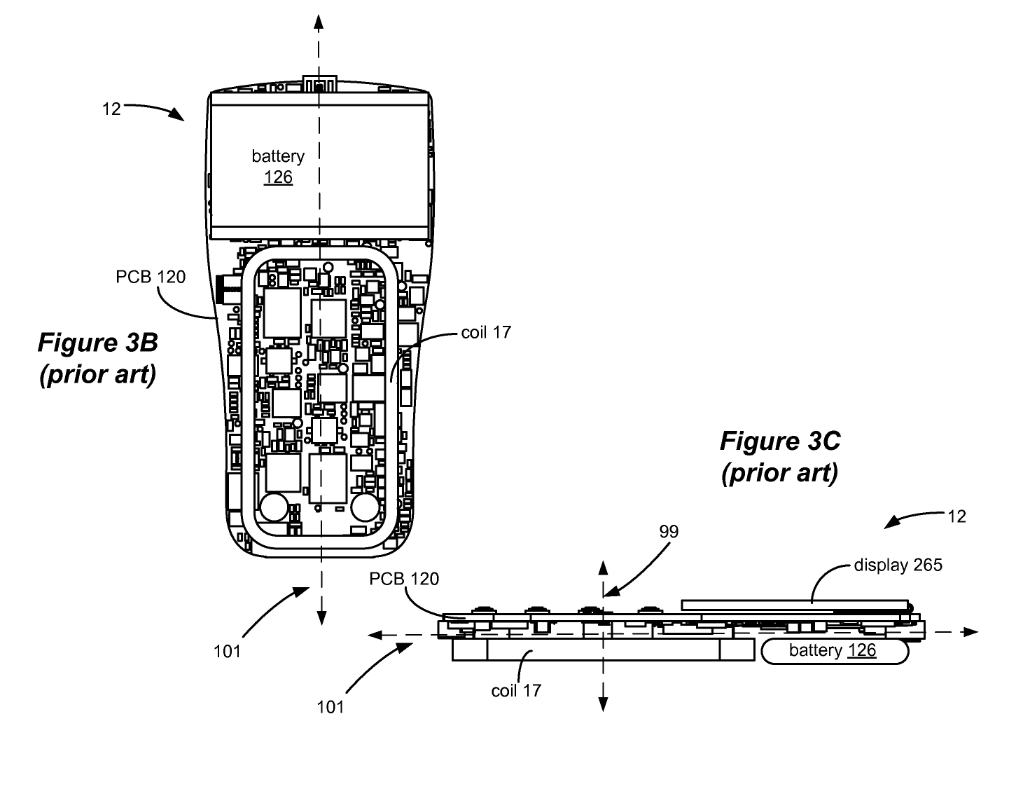

In one embodiment, illustrated in FIGS. 4A and 4B, an improved hand-held miniaturized remote controller 400 is small and light enough to be conveniently carried in a pocket or purse, or carried on a keychain or other similar device, but is large enough to be easily handled by a patient with limited hand flexibility. The embodiment illustrated in FIGS. 4A and 4B employs a housing 410 that is approximately 8.0 cm (3.15 in.) long, 3.5 cm (1.38 in.) wide at its widest, and 1.3 cm (0.51 in.) thick, but other sizes that are small relative to a conventional remote controller 12 such as shown in FIG. 3A, typically approximately 12.7 cm (5 in.) long, 5 cm (2 in.) wide, and 3 cm (1.2 in.) thick, can be used. The shape of the housing 410 is illustrative and by way of example only, and other shapes can be used.

The size of the housing 410 is not all that is constrained by the ability of the patient to use the miniaturized remote controller. Small buttons can be difficult for a patient to use. Generally, buttons should be no less than 19 mm wide (¾ in.), to allow a patient with poor eyesight, hand flexibility, or hand-eye coordination to press the desired button accurately. In one embodiment, the buttons 420 and 430 are 15 mm (0.39 in.) tall by 20 mm (0.79 in.) wide.

Because of the small size of the miniaturized remote controller 400, only a subset of the functionality of the user interface of a conventional remote controller 12 is provided by the user interface of the miniaturized remote controller 400. Although the user interface of the remote controller 12 has added functionality from generation to generation, the most frequently used functions on a conventional remote controller 12, are (a) turning the IPG 100 on and off, (b) increasing and decreasing the amplitude of the stimulation generated by the IPG 100, and (c) changing the program used by the IPG 100. In one embodiment, illustrated in FIG. 4A the only functions provided by the miniaturized remote controller 400 are to turn the IPG 100 on and off and to change the stimulation amplitude. Thus, the miniaturized remote controller 400 of FIG. 4A lacks the ability to change the program (e.g., timing, frequency, electrodes to be stimulated and their polarities, etc.) operating in the IPG 100.

Button 420 allows decreasing the amplitude of the stimulation, while button 430 allows increasing the amplitude. Button 440 allows turning the IPG 100 on or off. For protection against inadvertently turning the IPG 100 on or off, in some embodiments, button 440 can be recessed a small amount relative to a surface of the housing 410 and generally rounded with a diameter of about 10 mm. The shapes, arrangement, and the number of buttons illustrated in FIGS. 4A and 4B are illustrative and by way of example, and other shapes, arrangements, and number of buttons can be used. In addition, the miniaturized remote controller 400 can provide user interaction elements other than buttons, such as slide switches, rocker switches, and any other such element that can be used by patients with limited physical capability.

In addition to the user interaction elements, in the embodiments illustrated in FIGS. 4A/B, the miniaturized remote controller 400 provides an indicator light 450 to provide indications to the patient of related to the use of the miniaturized remote controller 400.

Other functions that are provided by a conventional remote controller such as the remote controller 12 of FIG. 3A-C can be added as desired, but in general, the miniaturized remote controller 400 has only a subset of the full functionality of the conventional remote controller 12, in order to allow the miniaturization. Thus, the patient typically needs both the conventional remote controller 12 in addition to the miniaturized remote controller 400 to achieve the full range of control over the IPG 100. The miniaturized remote controller 400 trades the reduced functionality for the convenience of having a remote controller that can be put on a keychain, in a pocket or purse, and carried by the patient continually without the bulk and weight of the conventional remote controller 12.

FIG. 4B illustrates the improved miniaturized remote controller 400 of FIG. 4A with an additional slide switch 460 that provides the patient the ability to change therapeutic programs for the IPG 100 by sliding the switch from one position to another. In the example illustrated in FIG. 4B, the slide switch 460 has two positions, one for a first stimulation program and the other for a second stimulation program, allowing the patient to choose between the two programs easily. Other embodiments can use a multi-position switch to provide the patient with the ability to select more than two programs. As shown in FIG. 4B, the positions of the switch 460 are labeled 1 and 2 to indicate the program selected. Other labeling or indicator techniques can be used, including, without limitation, additional indicator lights.

Alternatively, the miniaturized remote controller 400 in some embodiments uses an additional button similar to the button 420, to allow the patient to advance through a series of programs, with each button press switching to the next program. In another embodiment, two buttons can be used, one to select the previous program in the series of programs, and the other to select the next program in the series. Use of buttons instead of a slide switch typically takes additional space on the housing 410, and may require a larger housing 410 to accommodate the space needed for the buttons, thus reducing the size differential between the miniaturized remote controller 400 and the remote controller 12. Alternatively, such additional buttons could be placed on another surface of the housing 410, typically the side opposite the side illustrated in FIGS. 4A/B.

In some embodiments, buttons such as the buttons 420 and 430 illustrated in FIGS. 4A/B are made flush with the corresponding surface of the miniaturized remote controller 400, or can be positioned slightly recessed from the surface, to avoid or less the likelihood of inadvertent activation of the button if the miniaturized remote controller 400 is kept in, for example, a patient's pocket or purse.

In some embodiments, buttons can be manufactured to require a predetermined activation force, which provides tactile feedback to the patient, letting the patient know that the button has been pressed. In contrast, with the conventional remote controllers for the IPG 100, such as the remote controller 12 of FIG. 3A, as with conventional remote controls for other devices, no specific activation force is required to activate a button. In addition to providing tactile feedback, requiring a non-zero predetermined activation force also reduces the likelihood of inadvertent or unintended activation of the button. In some embodiments, the non-zero predetermined activation force is combined with recessing or making the buttons flush with a surface of the miniaturized remote controller, to further reduce the likelihood of inadvertent activation.

Another issue related to miniaturization of the remote controller is having a sufficient telemetry range, i.e., how far apart the IPG 100 and the miniaturized remote controller 400 can be and still successfully communicate with each other. The miniaturized remote controller 400 should have a telemetry range of at least 15 cm (6 in.) and preferably a range of 45 cm (18 in.) to 60 cm (24 in.), to allow the miniaturized remote controller 400 to transmit through the patient's body from front to back. This would allow positioning the miniaturized remote controller 400 in front of the patient, even when the IPG 100 is implanted at the back of the patient. The telemetry range requirement affects the choice of antenna and power source for the miniaturized remote controller 400.

In some embodiments, the miniaturized remote controller 400 uses a communications frequency of 100 KHz, selected to reduce the absorption of the radio frequency (RF) waves by the patient's body. In embodiments where absorption is less of an issue, such as where the IPG 100 is positioned at the front of the patient's body, higher frequencies, such as 400 MHz, can be used, with corresponding changes in the antenna.

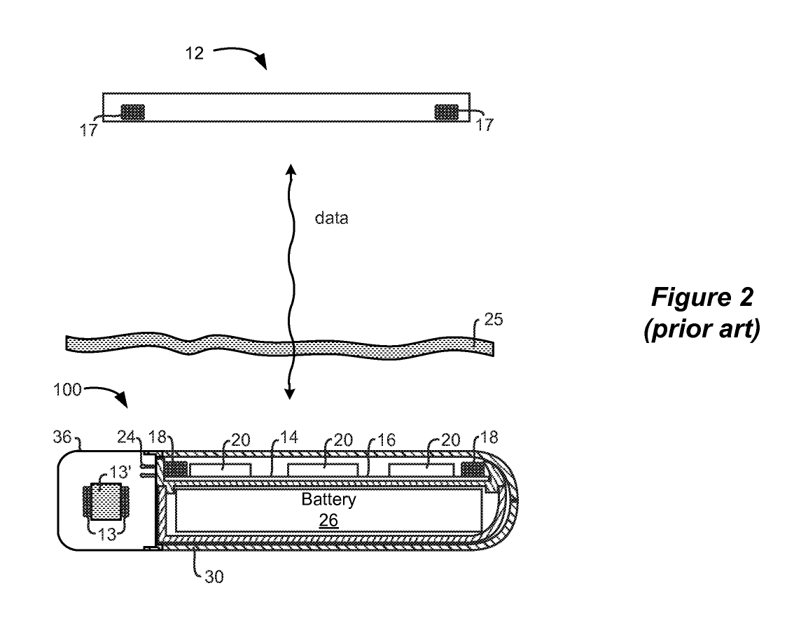

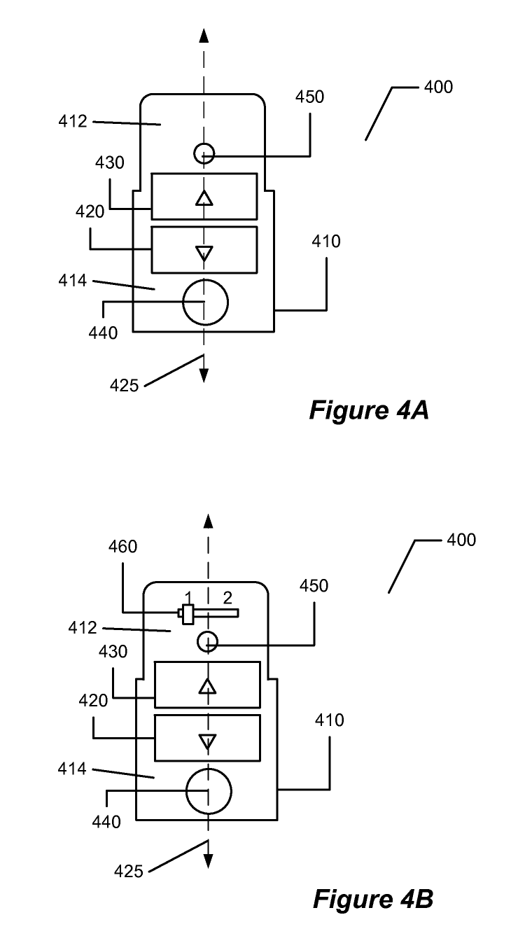

A conventional remote controller such as the remote controller 12 of FIG. 3B uses a flat air core coil antenna. Because of the smaller size of the miniaturized remote controller 400, an air core coil antenna such as the coil 17 of FIG. 2 could have difficulty to achieving the desired telemetry range. Instead, as illustrated in FIG. 5A-C, one embodiment uses a ferrite core antenna 510 oriented longitudinally with the housing 410. Alternatively, in some embodiments an air core antenna can be used, winding the antenna windings around the perimeter of the miniaturized remote controller housing. Such an antenna would be lighter and possibly more robust than a ferrite core antenna, but provides less help with the orientation of the antenna field.

The field of ferrite core antenna 510 is such that orientation of the miniaturized remote controller 400 relative to the IPG 100 is indicated to the patient by the shape of the miniaturized remote controller 400, which is generally shaped like a pointer. The ability to orient the miniaturized remote controller 400 just by feel can be useful, because the IPG 100 often is positioned in the patient in a place, such as above the buttocks, where the patient may not be able to see the miniaturized remote controller 400 when using it to control the IPG 100.

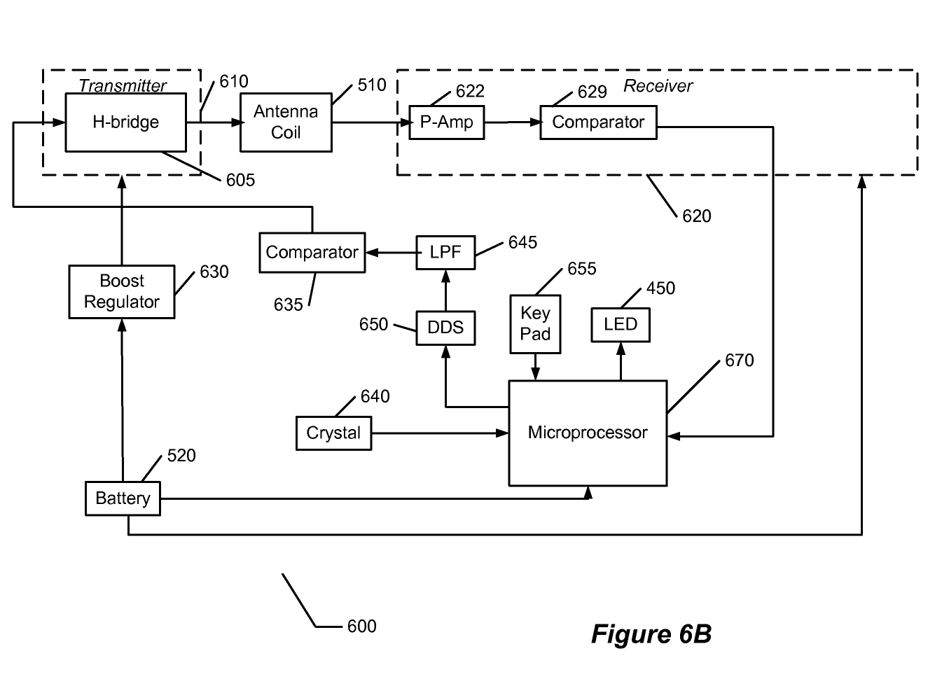

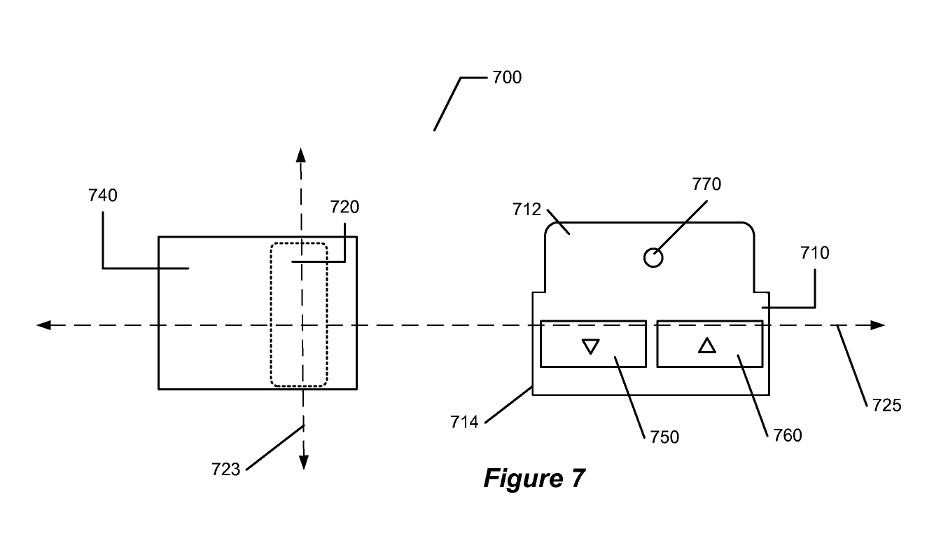

To provide such tactile feedback regarding orientation, in the embodiments illustrated in FIGS. 4A/B and 5, the housing 410 has two differently sized sections 412 and 414, which in this case such sections are aligned along the miniaturized remote controller 400's predominate axis 425. The differently sized sections 412 and 414, as well as this linear relationship of housing 410 comprising these sections, encourage a patient to hold the miniaturized remote controller 400 with the end of the smaller section 412 pointed at the IPG 100, which maximizes the telemetry range of the miniaturized remote controller 400 because of the longitudinal orientation of the antenna 510. In one embodiment, housing section 412 is narrower than housing section 414, but has the same thickness. Other configurations and shapes can be used to provide tactile feedback to the patient on the correct orientation of the miniaturized remote controller 400, as illustrated in FIGS. 7 and 8A/B.

FIG. 5B is a cutaway view along line A-A of the miniaturized remote controller 400 of FIG. 5A, further illustrating the relative positioning of a electronics package PCB 530, the battery 520, and the antenna 510, showing the ferrite core 540 and windings 550 of the antenna 510. FIG. 5C is a cutaway view along line B-B of the miniaturized remote controller 400 of FIG. 5A, further illustrating the positioning of the PCB 530, the battery 520, and the antenna 510. In the illustrated embodiment, the battery 520 and the PCB 530 are parallel to the axis 514 around which the coil of antenna 510 is wound, and the antenna 510 is offset along the line B-B from the PCB 530 and the battery 520.

Liquid crystal display (LCD) screens are common in conventional remote controllers, such as the LCD screen 265 of conventional remote controller 12 illustrated in FIG. 3A. In the miniaturized remote controller such as illustrated in FIGS. 4A/B, 7, and 8A/B, an LCD screen would take up excessive space. In addition, an LCD screen can cause interference with the reception or transmission of the miniaturized remote controller 400. Thus, the miniaturized remote controller 400 preferably omits a display, depending on visual or audible indicators, such as the indicator light 450, for patient feedback.

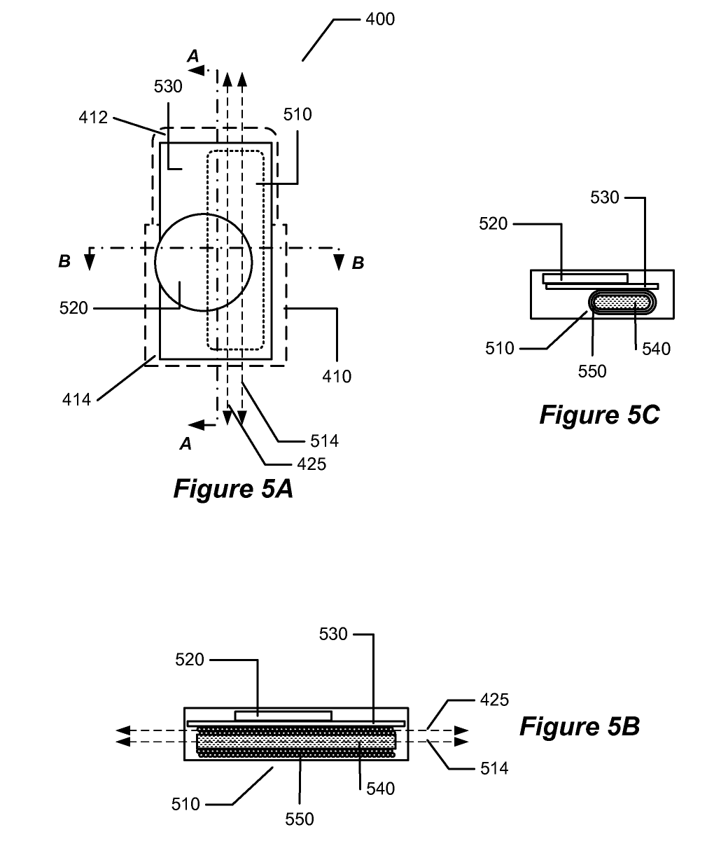

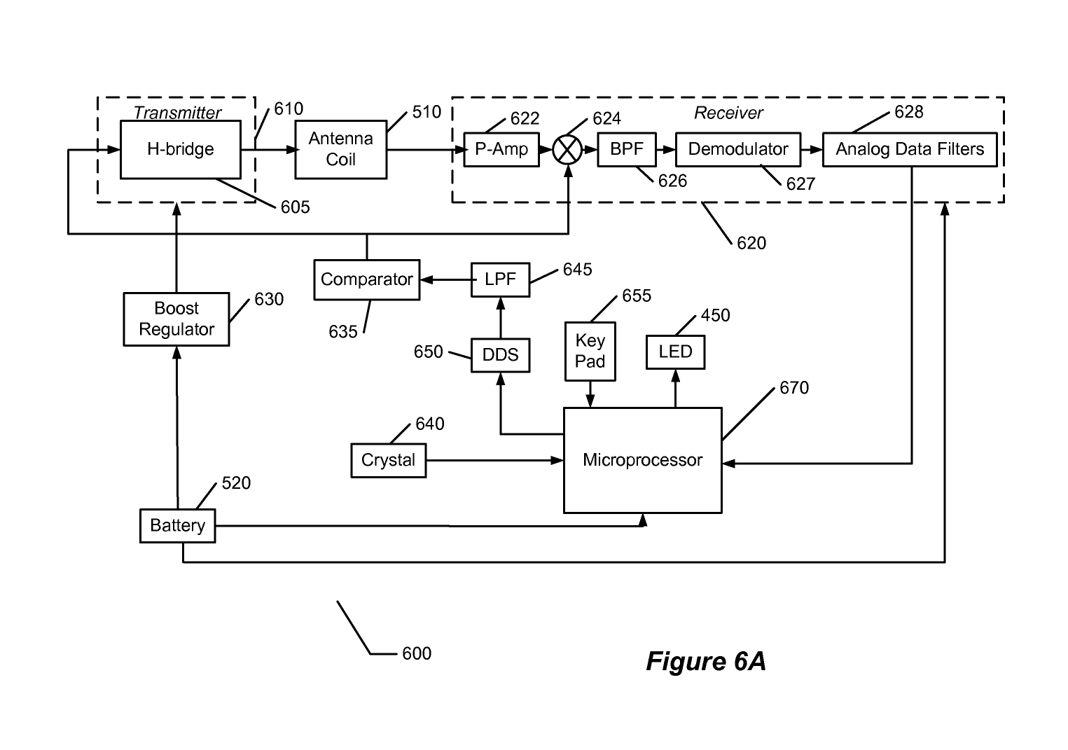

In a miniaturized remote controller, interference between elements such as the electronics package 530 and the antenna 510 can occur. To reduce such interference, embodiments of the miniaturized remote controller avoid axial alignment of the electronics package PCB 530 and the antenna 510, as shown in FIGS. 5B/C. In addition, in some embodiments, portions of the circuitry 600 (FIGS. 6A/B) contained in the electronics package PCB 530 that are unnecessary for reception are shut down or de-powered during reception, to further reduce interference or noise in the received signal.

Because of the need for convenience and portability, there is a strong preference for a battery-powered miniaturized remote controller 400. In one embodiment, illustrated in Fig. 5A-C, the battery 520 is a non-replaceable battery designed for recharging in the miniaturized remote controller 400. In a more preferred embodiment, the battery 520 is a replaceable battery. Although the replaceable battery can be disposable or rechargeable, the miniaturized remote controller 400 typically does not provide for in-place recharging of the battery. In one embodiment, a commonly available coin or button-type battery is used, such as a CR2025 lithium battery from multiple manufacturers. Although other, less commonly available replaceable batteries can be used, such batteries are less preferred because of the difficulty a patient may have in finding a replacement battery, while button cells such as a CR2025 battery can be obtained in a wide variety of retail outlets, making quick replacement relatively easy. The housing 410 in such an embodiment has an opening (not shown) for installation of the battery 520 and a cover (not shown) designed for a patient with limited physical capacity to open the miniaturized remote controller 400 easily and replace the battery 520 without the use of tools.

In embodiments with a rechargeable-in-place battery, the remote controller 400 can use any technique known in the art for recharging, including exposed leads, a USB port, or inductive charging. All such recharging techniques require additional space in the remote controller for recharging circuitry. In addition, when using a recharging technique other than inductive recharging, an electrostatic discharge could enter the recharge port, causing damage to the miniaturized remote controller 400 and possibly affecting the stimulation delivered by the IPG 100. Furthermore, a rechargeable battery can be less convenient for the patient, because of the potential need for carrying around recharging components, as well as the time needed to recharge the rechargeable battery. In contrast, a replaceable battery can be removed from the miniaturized remote controller 400 and replaced faster than recharging the battery, and avoids the need for an additional recharger and a power source for the recharger. Although as shown in FIGS. 5A-C a single button cell battery is used, in some embodiments, multiple button cells or other batteries, connected in series or parallel, can be used as a power source if desired.

As shown in FIGS. 4A and 4B, only a limited amount of status feedback is available on the miniaturized remote controller 400. In the illustrated embodiments, a single indicator light 450, typically an light emitting diode (LED), provides an indication of the charge status of the IPG 100 and whether the miniaturized remote controller 400 can communicate with the IPG 100. Preferably, the indicator light 450 is a multi-color LED, to use color as part of the indication, in addition to the on-off status of the LED. In some embodiments, the miniaturized remote controller 400 will flash or blink the indicator light 450 to indicate certain conditions, and be solid on or off to indicate other conditions.

In one embodiment, the indicator light 450 provides an indication of multiple status information simultaneously. A solid green light for three seconds after a button press indicates that the IPG 100 successfully received a message from the miniaturized remote controller 400 and that the battery of the IPG 100 has an acceptable charge level. If the indicator light 450 is yellow instead of green, the message was successfully received, but the IPG 100 battery has an unacceptably low charge level and should be recharged. Recharging the IPG 100 battery is typically not a feature of the miniaturized remote controller 400, and the conventional remote controller 12 or an external recharging unit is used for recharging IPG 100, because the size of the miniaturized remote controller 400 does not provide sufficient room for the additional recharging coils and circuitry.

If the miniaturized remote controller 400 failed to communicate with the IPG 100, for example because the miniaturized remote controller 400 was too far away from the IPG 100 or was incorrectly oriented, then the indicator light 450 blinks yellow in some embodiments. In one embodiment, the indicator 450 blinks at a 3 Hz rate for 10 seconds. During that time, the miniaturized remote controller 400 automatically and repeatedly retries communication with the IPG 100. If the miniaturized remote controller 400 is successful during the 10 second retry period, then the indicator light turns to solid green or yellow (depending on the charge level of the IPG 100, as described above) for five seconds, to indicate a successful retry. The 10-second retry period allows the patient to move the miniaturized remote controller 400 closer to or in better alignment with the IPG 100.

In some embodiments, the indicator 450 blinks at a different rate, for example, 1 Hz, for five seconds when the battery 520 is inserted into the miniaturized remote controller 400, to indicate a good battery 520 has been inserted properly into the miniaturized remote controller 400 and the miniaturized remote controller 400 is functioning properly. In such embodiments, the indicator light does not indicate any other status information for the miniaturized remote controller 100.

The colors, frequencies of blinking, and time periods described above are illustrative and by way of example only. Other colors, frequencies, and time periods, and other uses of the indicator 450 to indicate IPG 100 status, communication events, and the miniaturized remote controller 400 status can be provided as desired. For example, additional indicator lights can be used in some embodiments to indicate the program being used by the IPG 100, or one indicator can be used to indicate an attempt to communicate with the IPG 100 and a second indicator used to show success or failure of the attempt. Alternatively, other indicator lights, such as one or more lights that indicate which program is being used by the IPG 100 can be placed in the housing 410, at the cost of additional space on the miniaturized remote controller 400, typically increasing its size. One of skill in the art will recognize that additional housing space on a surface of the miniaturized remote controller 400 may be desirable for labels indicating the purpose of the indicator light or lights in some embodiments, particularly if more than one indicator is used.

In addition, in some embodiments a sound generator can be included, in addition to or instead of the indicator light 450, to provide auditory feedback by emitting beeps or other kinds of sounds to indicate the result of the use of the miniaturized remote controller 400 or the status of the IPG 100. For example, the sound generator can generate a sustained sound at either a first or second predetermined pitch frequency to indicate a successful communication with the IPG 100 and its charge level, and an intermittent sound can indicate an unsuccessful attempt and the need to retry. The above uses of a sound generator are illustrative and by way of example only, and other techniques for audibly indicating the result of user activation of the miniaturized remote controller 400 can be used. In other embodiments, the sound generator can generate a click or other predetermined sound to provide auditory feedback a button has been pressed, in addition to or instead of providing the feedback described above. Use of auditory feedback can allow a blind or partially blind patient to use the miniaturized remote controller 400, even without being able to see the indicator light 450. However, such a sound generator takes up additional space in the miniaturized remote controller 400 and typically would increase the size of the miniaturized remote controller 400.

In the illustrated embodiments, no indicator is provided for the battery status of the miniaturized remote controller 400 (other than at insertion time) or for the on-off status of the IPG 100. These indicators are preferably omitted from the miniaturized remote controller 400 to conserve space and to make the miniaturized remote controller 400 easier to use compared to the patient's otherwise standard fully functional remote controller 12. The full-sized remote controller 12 can provide those indications if desired. Similarly, the patient can determine that the miniaturized remote controller 400 battery needs replacement or recharging when the miniaturized remote controller 400 stops functioning and the indicator 450 dims or darkens.

Although having only a single indicator light 450 limits the amount of information that can be communicated to the patient by the miniaturized remote controller 400, some patients find that the variety of information provided by a conventional remote controller 12 confusing and difficult to use. Thus, contrary to the usual practice of providing more information, the limited information capability of the miniaturized remote controller 400 can be advantageous to some patients.

FIG. 6A is a block diagram illustrating one embodiment of the circuitry 600 for a miniaturized remote controller 400 according to one embodiment. A microprocessor 670 provides processing logic for the miniaturized remote controller 400, controlling the various features and functions of the miniaturized remote controller 400, including programming the miniaturized remote controller 400 by a programming unit (not shown). Power to the miniaturized remote controller 400 is provided by the battery 520, as described above. An antenna coil 510, typically a ferrite core antenna, as described above, allows for communication with the IPG 100, as well as with the programming device.

A crystal 640 provides clocking for the microprocessor 670. An LED 450 or other suitable indicator light provides feedback to the patient about the results of interaction with the miniaturized remote controller 400, as described above. Patient interaction is typically through a key pad 655, which provides an interface to the buttons 420, 430, and 440, and in embodiments providing for selecting between multiple stimulation programs in the IPG 100, the slide switch 460 or other selection circuitry, such as shown in FIG. 4B and described above. A transmitter 610 is powered from the battery 520, and includes an H-bridge 605 driving the antenna 510 by the output of a comparator 635. The sinusoidal wave output of a direct digital synthesizer 650 is filtered by a low pass filter 645, then digitized to two levels by the comparator 635.

As described above, the antenna 510 is also connected to a receiver 620, which receives signals from the antenna 510. A pre-amp 622 amplifies the signals, which are mixed by mixer 624 under the control of the comparator 635, producing an amplified signal that is passed through a band pass filter 626 to a demodulator 627 and then to analog data filters 628, which are used by the microprocessor 670 to determine whether the message sent to the IPG 100 was successfully received, as well as for receiving programming instructions from the programming unit.

FIG. 6B is a block diagram illustrating an alternate embodiment of the circuitry 600 for a miniaturized remote controller 400, which allows for reducing the size of the remote controller 400 by reducing the number of circuitry parts used. As illustrated in FIG. 6B, the mixer 624, the band pass filter 626, the demodulator 627, and the analog data filters 628 of the receiver 620 are replaced by a comparator 629. The microprocessor 670 further performs software demodulation of signals received from the comparator 629 to determine whether the message sent to the IPG 100 was successfully received, as well as for receiving programming instructions from the programming unit.

Any desirable protocol can be used to communicate between the miniaturized remote controller 400 and the IPG 100. The miniaturized remote controller 400 typically receives an acknowledgement message from the IPG 100, allowing the miniaturized remote controller 400 to determine whether the transmitted data were successfully received by the IPG 100. In some embodiments, the acknowledgement from the IPG 100 can be included as part of another transmission from the IPG 100. Because the miniaturized remote controller 400 is intended for use as a complement to a full-sized remote controller, the same communication protocol is used in the miniaturized remote controller 400 as in the corresponding full-sized remote controller.

Typically, each message sent to the IPG 100 includes an error checking code such as a cyclical redundancy code (CRC), to ensure data integrity. In such embodiments, the IPG 100 recalculates the CRC and compares it to the CRC contained in the transmission, indicating an error if the CRCs fail to match, usually by requesting a retry of the communication. Other transmission techniques known to the art, such as using error-correction codes (ECCs) can be used. In addition to error indications returned by the IPG 100, failure to receive an acknowledgment from the IPG 100, typically after a timeout period as described above, can indicate an unsuccessful receipt of the message from the miniaturized remote controller 400. Such a situation can occur where the patient incorrectly positions or orients the miniaturized remote controller 400, such as positioning it outside the telemetry range of the miniaturized remote controller 400.

Remote controllers for an IPG 100 are typically programmed to associate them with a specific IPG 100, so that one patient's remote controller is not usable to modify the stimulation of another patient's IPG 100. In conventional remote controllers, such as the remote controller 12 of FIGS. 3A-C, the preferred programming technique uses a USB port connected to a programming device. In the miniaturized remote controller 400, a USB port is less preferred, because of the additional space required for the USB connector and associated circuitry. Similarly, use of an infrared (IR) port is less preferred because of the space requirement, as well as because of problems that can be caused by dirt or other substances obscuring the IR port, interfering with reception by the miniaturized remote controller 400.

Instead, one embodiment uses the antenna 510 to receive programming instructions from a wireless programming device, which can comprise the full-sized remote controller 12, in addition to using the antenna 510 to transmit instructions to the IPG 100. The processing logic 670 can interpret signals received over the antenna 510 as a plurality of programming instructions, which are then used to program the processing logic 670. In programming mode, the antenna 510 and processing logic 670 operate as a slave to the master programming device. In operational mode, the antenna 510 and the processing logic 670 operate as a master to the IPG 100. The programming technique and communication protocols used by conventional remote controllers and programming devices can be used to program the miniaturized remote controller 400, and in some embodiments, the same programming device can be used to program both the miniaturized remote controller 400 and a full-sized remote controller, even though the full-sized remote controller 12 can be connected using a wired connection and the miniaturized remote controller 400 can be connected wirelessly.

FIGS. 7 and 8 illustrate some alternate embodiments of a miniaturized remote controller. FIG. 7 illustrates two views of an embodiment of a miniaturized remote controller 700 where the antenna 720 is wound around an axis 723 perpendicular to a long axis 725 of the housing 710. As with the miniaturized remote controller 400, the miniaturized remote controller 700 uses a housing 710 with two sections 712 and 714, where section 712 is narrower than section 714, encouraging the patient to aim the miniaturized remote controller 700 with the narrower section toward the IPG 100. Buttons 750 and 760 are oriented parallel to the long axis 725 of housing 710 and aligned with each other along that dimension, further providing a tactile indication of the proper orientation. Indicator 770 (e.g., an LED) is positioned in the smaller section 712, but could be positioned elsewhere as desired.

FIG. 8A/B is a block diagram illustrating yet another embodiment of a miniaturized remote controller 800. In FIG. 8A, a perspective view of the housing 810 illustrates a largely rectangular cross-section, but other cross-sectional configurations can be used. Like antenna 510 of FIG. 5A, antenna 810 is oriented longitudinally in the housing 810, which offers by its elongated shape alone a tactile feedback to the patient to help the patient orient the miniaturized remote controller 800 correctly towards the IPG 100. A battery 830, which can be a stacked plurality of batteries, is inserted into the housing 810 at one end of the housing 810. In one embodiment, the housing 810 provides a slide or flap closure for a battery compartment (not shown) for holding the battery 830 and electrically connecting it to electronics in the miniaturized remote controller 800.

FIG. 8B is a top view of top surface 815 of the housing 810, which contains an on/off button 840, a rocker switch 850, and an indicator light 860. The on/off switch 840 behaves as the button 440 of miniaturized remote controller 400 in FIG. 4A/B to turn the IPG 100 on or off. In another embodiment, the button 840 can be used to cycle between multiple IPG 100 programs, with one position in the cycle turning off the IPG 100, activating the button again to turn the IPG 100 on or to cycle to the next program. The rocker switch 850 can be pressed on one end to increase stimulation amplitude and on the other end to decrease stimulation amplitude. The indicator light 860 behaves as the indicator light 450 of FIG. 4A/B. Although shown as aligned with the button 840 and the rocker switch 850 in FIG. 8B, the indicator light can be mounted on another surface, including an end surface, of the housing 810 as desired. The user interaction elements 840 and 850 can also be arranged on different surfaces from each other instead of being arranged together as shown in FIG. 8A/B.

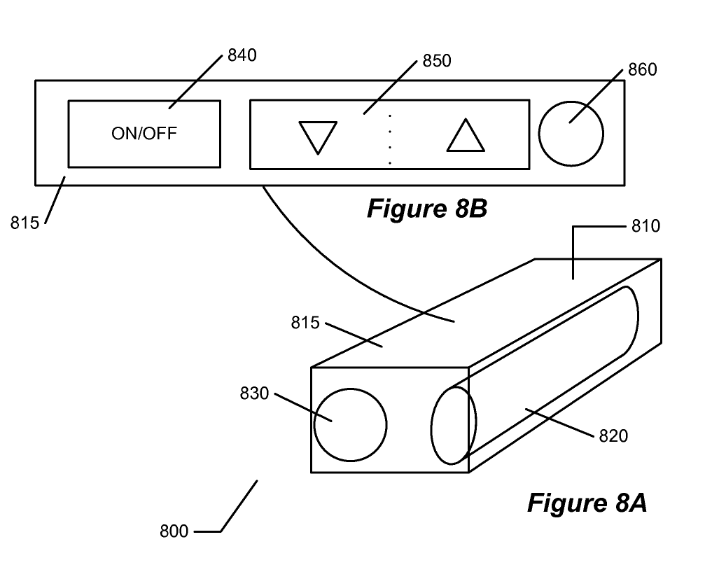

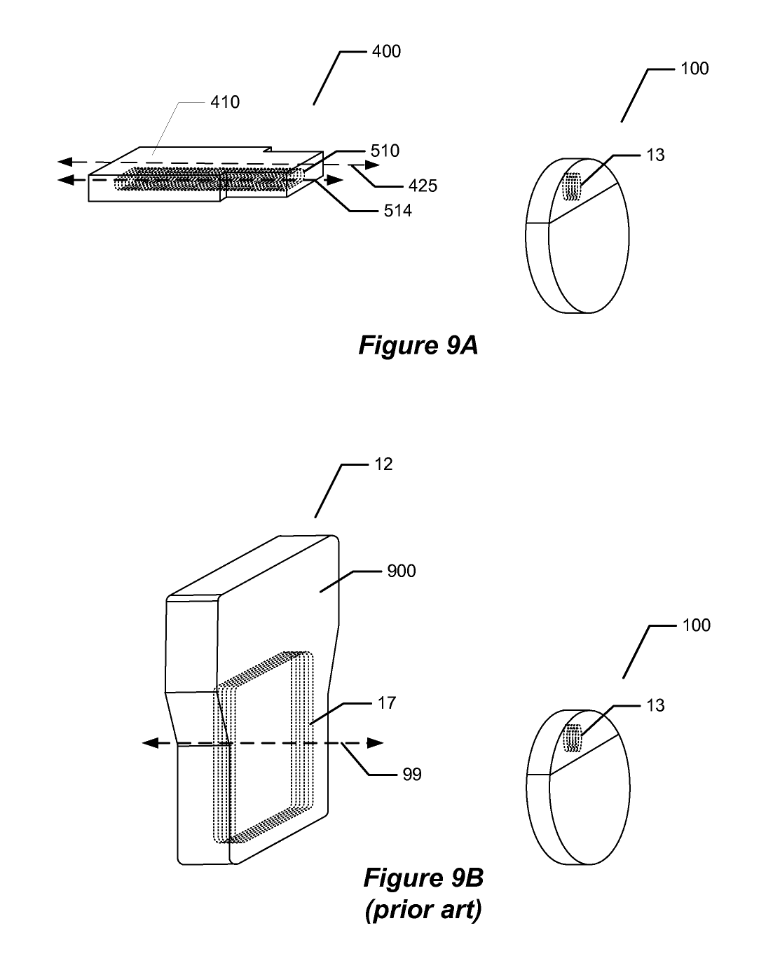

FIGS. 9A/B compare the orientation of the improved miniaturized remote controller 400 and the conventional remote controller 12 relative to the IPG 100 when used to communicate with the IPG 100. In FIG. 9A, the miniaturized remote controller 400 is oriented with the long axis 425 of the housing 410 aimed at the coil 13 of the IPG 100, which can be achieved by feel because of the shape of the housing 410. This tactilely assisted orientation aims the axis 514 of the antenna 510 at the coil 13, maximizing the field strength at the coil 13.

In contrast, as illustrated in FIG. 9B, with the conventional remote controller 12, the patient positions the large flat side 900 of the conventional remote controller 12 roughly parallel to the relatively flat IPG 100 to orient the axis 99 of the coil 17 relative to the coil 13, and the shape of the housing of the conventional remote controller 12 does not tactilely indicate how to aim the remote controller 12 at the IPG 100.

While certain example embodiments have been described in details and shown in the accompanying drawings, it is to be understood that such embodiments are merely illustrative of and not devised without departing from the basic scope thereof, which is determined by the claims that follow. Various changes and modifications may be made without departing from the spirit and scope of the present invention. Thus, alternatives, modifications, and equivalents may fall within the spirit and scope of the present invention as defined by the claims. By way of example and not limitation, the specific electrical components utilized may be replaced by known equivalents or other arrangements of components that function similarly and provide substantially the same result.

We claim:

1. A system comprising:
an implantable medical device,
a first hand held remote controller for communicating with the implantable medical device, the first remote controller having a housing of a first size and comprising a first user interface and for performing a first set of functions; and
a second hand held remote controller for communicating with the implantable medical device, the second remote controller having a housing of a second size and comprising a second user interface and for performing a second set of functions,
wherein the second size is smaller than the first size, and
wherein the second set of functions comprises a subset of the first set of functions.

2. The system of claim 1,
wherein the first user interface comprises a display, and
wherein the second user interface does not comprise a display.

3. The system of claim 1, wherein the second user interface comprises:
a first user interaction element, disposed with the housing, configured to select one of a plurality of therapeutic program for the implantable medical device;
a second and a third user interaction element, disposed with the housing, configured to control a stimulation amplitude of the implantable medical device;
a fourth user interaction element, disposed with the housing, configured to turn the implantable medical device on and off; and
an indicator light disposed with the housing.

4. The system of claim 1, wherein the second set of functions comprises:
modifying the amplitude of a stimulation generated by the implantable medical device; and
turning the implantable medical device on and off.

5. The system of claim 4, wherein the second set of functions further comprises:
selecting one of a plurality of therapeutic programs for the implantable medical device.

6. The system of claim 1, wherein the second remote controller comprises an antenna, configured to communicate with the implantable medical device and a programming unit.

7. A remote controller for wirelessly communicating with an implantable medical device, comprising:
a housing having a longest dimension along a long axis; and
a coil within the housing adapted for sending and receiving communications to and from the implantable medical device,
wherein the coil is wrapped around a coil axis parallel to the long axis of the housing.

8. The remote controller of claim 7, wherein the housing comprises:
a first section and a second section positioned along the long axis, wherein the first section is sized differently from the second section.

9. The remote controller of claim 7, wherein the housing comprises a first section and a second section positioned along the long axis of the housing, wherein the first section is sized differently from the second section.

10. The remote controller of claim 7, wherein the housing has an elongated shape configured to provide a tactile feedback for indicating a correct orientation of the remote controller relative to the implantable medical device.

11. The remote controller of claim 7, further comprising a user interface for indicating status information.

12. The remote controller of claim 11,
wherein the user interface comprises an indicator light, and
wherein the indicator light is controlled to indicate success or failure of a communication with the implantable medical device.

13. A remote controller for wirelessly communicating with an implantable medical device, comprising:
a user interface for indicating status information to a user, wherein the user interface comprises an indicator light, and
wherein the indicator light is controlled to indicate a first condition, comprising success or failure of a communication with the implantable medical device.

14. The remote controller of claim 13, wherein the indicator light is further controlled to indicate a retry period for retrying a failed communication with the implantable medical device.

15. The remote controller of claim 13, wherein the indicator light is further controlled to indicate a second condition, comprising a status of the implantable medical device.

16. The remote controller of claim 15, wherein the first condition and the second condition are simultaneously indicated by the indicator light.

17. The remote controller of claim 13, further comprising a replaceable battery,
wherein the indicator light is further controlled to indicate a status of the battery upon replacement.

18. The remote controller of claim 13, further comprising:
a user interaction element, configured to trigger a communication with the implantable medical device, wherein the user interaction element is configured to activate upon a predetermined activation force on the user interaction element.

19. A remote controller for wirelessly communicating with an implantable medical device, comprising:
a housing having an axis, wherein the housing comprises a first section and a second section positioned along the axis of the housing, wherein the first section is sized differently from the second section; and
a coil within the housing for sending and receiving communications to and from the implantable medical device, wherein the coil is wrapped around a coil axis parallel to the long axis of the housing.

20. The remote controller of claim 19, further comprising: a user interface for indicating status information to a user, comprising an indicator,
wherein the indicator is controlled to indicate a first condition, comprising success or failure of a communication with the implantable medical device

21. The remote controller of claim 20, wherein the indicator is an indicator light.

22. The remote controller of claim 20, wherein the indicator is a sound generator.

23. The remote controller of claim 20, wherein the indicator light is further controlled to indicate a second condition, comprising a status of the implantable medical device.

24. The remote controller of claim 20, wherein the first condition and the second condition are simultaneously indicated by the indicator.
